(19) United States
(12) Patent Application Publication
(10) Pub. No.: US 2024/0102992 A1
(43) Pub. Date: Mar. 28, 2024

(54) MEASUREMENT METHOD FOR ANTIBODY-DEPENDENT CELLULAR CYTOTOXICITY ACTIVITY

(71) Applicant: NIPPON ZENYAKU KOGYO CO., LTD., Koriyama-shi (JP)

(72) Inventors: Takuya MIZUNO, Yamaguchi-shi (JP); Masaya Igase, Yamaguchi-shi (JP); Yoshiho Takeda, Yamaguchi-shi (JP)

(73) Assignee: NIPPON ZENYAKU KOGYO CO., LTD., Koriyama-shi (JP)

(21) Appl. No.: 18/366,822

(22) Filed: Aug. 8, 2023

(30) Foreign Application Priority Data
Aug. 9, 2022 (JP) 2022-126770

(51) Int. Cl. G01N 33/50; C07K 14/705; C07K 14/735; G01N 33/58

(52) U.S. Cl. CPC G01N 33/5011; C07K 14/70535; C07K 14/70596; G01N 33/581; C07K 2319/00

(57) ABSTRACT

A low-variation and highly reproducible method for measuring antibody-dependent cellular cytotoxicity activity of an antibody for a dog as the subject of administration is provided.

A method for measuring antibody-dependent cellular cytotoxicity activity comprising a step of causing an antibody and cells in which a canine CD16-canine Fcε receptor γ chain fusion protein is expressed in human-derived NK cells to act on canine tumor-derived cells or cells which express a canine tumor antigen and a step of detecting the cell death of the canine tumor-derived cells or the cells which express the canine tumor antigen. Also, the method for measuring antibody-dependent cellular cytotoxicity activity in which the human-derived NK cells are NK-92 cells.

### REFERENCE TO SEQUENCE LISTING SUBMITTED VIA PATENT CENTER

This application is being filed electronically via patent Center and includes an electronically submitted sequence listing in .xml format. The .xml file contains a sequence listing entitled “ZENOAQ-4-US.xml” created on Jul. 27, 2023 and is 24,110 bytes in size. The sequence listing contained in this .xml file is part of the specification and is hereby incorporated by reference herein in its entirety.

### TECHNICAL FIELD

The present invention relates to a method for measuring antibody-dependent cellular cytotoxicity activity. More specifically, the invention relates to a method for measuring antibody-dependent cellular cytotoxicity activity on a canine tumor.

### BACKGROUND ART

For tumor therapy, various antibody drugs are developed, and it is essential to measure antibody-dependent cellular cytotoxicity (ADCC) (sometimes simply referred to as ADCC below) activity for assessing the functions thereof.

For example, Patent Document 1 discloses a GPC3-targeting drug containing an anti-GPC3 antibody having antibody-dependent cellular cytotoxicity (ADCC) activity and/or complement-dependent cytotoxicity (CDC) activity as a therapeutic agent which is administered to a cancer patient. Moreover, Patent Document 2 discloses a native monoclonal antibody which induces ADCC in a combination therapy agent for treating a cancer.

As described above, a technique for measuring ADCC activity and thus obtaining an antibody drug which is found to have a sufficient function has been established for tumor therapy for a human as the subject of administration, but no satisfying technique has been established yet for antibody drugs for a non-human animal, such as a dog, as the subject of administration.

Antibody-dependent cellular cytotoxicity is induced when FcγIIIa receptor (which is also known as CD16) on a natural killer cell (sometimes simply referred to as an NK cell below) recognize the Fc region of an antibody. With a canine NK cell, however, ADCC is not necessarily induced.

Accordingly, for an antibody drug for a dog as the subject of administration, canine primary peripheral blood mononuclear cells (PBMC cells) called lymphokine-activated killer cells (LAK cells) or PBMC cells stimulated with interleukin-2 (IL-2) have been used for measuring ADCC activity (for example, Non-Patent Documents 1 to 4 and the like). However, it is required to prepare these cells for each ADCC activity measurement, and there is thus a problem because the properties of the cells vary and because reproducible results cannot be obtained.

Therefore, the present inventors have tried to provide a low-variation and highly reproducible method for measuring antibody-dependent cellular cytotoxicity activity of an antibody for a dog as the subject of administration.

### CITATION LIST

### Patent Literature

Patent Document 1: WO2015/170480
Patent Document 2: JP6748105B

### Non Patent Literature

Non-Patent Document 1: Kim, Y., Lee, S. H., Kim, C. J., Lee, J. J., Yu, D., Ahn, S., Shin, D. J., Kim, S. K., 2019. Canine non-B, non-T NK lymphocytes have a potential antibody-dependent cellular cytotoxicity function against antibody-coated tumor cells. BMC Vet. Res. 15, 339.
Non-Patent Document 2: Mizuno, T., Kato, Y., Kaneko, M. K., Sakai, Y., Shiga, T., Kato, M., Tsukui, T., Takemoto, H., Tokimasa, A., Baba, K., Nemoto, Y., Sakai, O., Igase, M., 2020. Generation of a canine anti-canine CD20 antibody for canine lymphoma treatment. Sci. Rep. 10, 11476.
Non-Patent Document 3: Oyamada, T., Okano, S., 2020. Cytotoxicity effect of trastuzumab on canine peripheral blood mononuclear cells. Iran J. Vet. Res. 21, 263-268.
Non-Patent Document 4: Strietzel, C. J., Bergeron, L. M., Oliphant, T., Mutchler, V. T., Choromanski, L. J., Bainbridge, G., 2014. In vitro functional characterization of feline IgGs. Vet. Immunol. Immunopathol. 158, 214-223.

### SUMMARY OF INVENTION

### Technical Problem

An object of the invention is to provide a low-variation and highly reproducible method for measuring antibody-dependent cellular cytotoxicity activity of an antibody for a dog as the subject of administration.

### Solution to Problem

As a result of intensive examination to solve the problems of the invention, the present inventors have produced cells in which a canine CD16-canine Fc ε receptor γ chain fusion protein was expressed in human-derived NK cells. Then, the inventors have found that antibody-dependent cellular cytotoxicity activity can be measured with low variation and high reproducibility when an antibody and the cells are caused to act on canine tumor-derived cells or cells which express a canine tumor antigen, and the invention has been thus completed.

That is, the invention relates to the method for measuring antibody-dependent cellular cytotoxicity activity and the like shown in (1) to (12) below.

(1) A method for measuring antibody-dependent cellular cytotoxicity activity comprising a step of causing an antibody and cells in which a canine CD16-canine Fc ε receptor γ chain fusion protein is expressed in human-derived NK cells to act on canine tumor-derived cells or cells which express a canine tumor antigen and a step of detecting the cell death of the canine tumor-derived cells or the cells which express the canine tumor antigen.

(2) The measurement method according to (1) above, wherein the human-derived NK cells are NK-92 cells.

(3) The measurement method according to (1) or (2) above, wherein the cells which express the canine tumor antigen are lymphoma-derived cells containing a labeling substance.

(4) The measurement method according to any of (1) to (3) above, wherein the canine tumor antigen is CD20.

(5) The measurement method according to (3) or (4) above, wherein the labeling substance is luciferase.

(6) The measurement method according to any of (3) to (5) above, wherein the lymphoma-derived cells are A20 cells or EL-4 cells.

(7) The measurement method according to any of (1) to (6) above, wherein the antibody is an antibody for the canine tumor antigen and is an antibody having antibody-dependent cellular cytotoxicity activity.

(8) The measurement method according to (7) above, wherein the antibody is an anti-canine CD20 antibody.

(9) Cells for measuring antibody-dependent cellular cytotoxicity activity in which a canine CD16-canine Fc ε receptor γ chain fusion protein is expressed in human-derived NK cells.

(10) A method for producing the cells according to (9) above, comprising a step of introducing a gene encoding canine CD16 and a gene encoding canine Fc ε receptor γ chain into the human-derived NK cells.

(11) Cells for measuring antibody-dependent cellular cytotoxicity activity in which canine CD20 and a labeling substance are expressed in lymphoma-derived cells.

(12) A method for producing the cells according to (11) above, comprising a step of introducing a gene encoding canine CD20 and a gene encoding the labeling substance into the lymphoma-derived cells.

### Advantageous Effects of Invention

According to the invention, a low-variation and highly reproducible method for measuring antibody-dependent cellular cytotoxicity activity also of an antibody for a dog as the subject of administration can be provided. Moreover, when the measurement method of the invention is used, an antibody drug which is useful for tumor therapy for a dog as the subject of administration is easily developed.

### BRIEF DESCRIPTION OF DRAWINGS

FIG. 1 A schematic figure of a plasmid encoding a fusion protein of canine CD16 and canine Fcs receptor γ chain is shown.

FIG. 2 is a figure showing the vector map of pMx-luc-IH (Example 2).

FIG. 3 is a figure showing the cell-mediated cytotoxicity activities using cell lines expressing a canine tumor antigen as the labeled cells and NK-92/cCD16γ cell line as the effector cells (Example 3).

FIG. 4 is a figure showing the cell-mediated cytotoxicity activities using A20/cCD20/luc cell line as the target cells and NK-92 cell line or NK-92/cCD16γ cell line as the effector cells (Example 3).

FIG. 5 is a figure showing the antibody concentration-dependent cell-mediated cytotoxicity activities using A20/cCD20/luc cell line as the target cells and NK-92 cell line as the effector cell line (Example 3).

### DESCRIPTION OF EMBODIMENTS

The “method for measuring antibody-dependent cellular cytotoxicity activity” of the invention means a method which comprises a step of causing “an antibody” and “cells in which a canine CD16-canine Fc ε receptor γ chain fusion protein is expressed in human-derived NK cells” to act on “canine tumor-derived cells or cells which express a canine tumor antigen” and which comprises a step of detecting the cell death of “the canine tumor-derived cells or the cells which express the canine tumor antigen”. The “method for measuring antibody-dependent cellular cytotoxicity activity” may comprise another step which is useful for measuring antibody-dependent cellular cytotoxicity activity as long as the method comprises these steps.

Here, the “canine tumor-derived cells or cells which express a canine tumor antigen” refer to the target cells of the antibody. The “canine tumor-derived cells” may be cells which are directly collected from a canine tumor or cells obtained by culturing collected cells.

Moreover, the “cells which express a canine tumor antigen” mean cells in which a gene encoding CD20, CD19, CD70 or the like, which are known as canine tumor antigens, is incorporated and expressed in cells derived from lymphoma or the like. The cells may be any cells as long as the cells can express a canine tumor antigen, and examples thereof comprise lymphoma-derived cells of a human, a dog, a mouse or the like. Examples of mouse lymphoma-derived cells comprise A20 cells, EL-4 cells and the like.

The “cells which express a canine tumor antigen” preferably further contain a labeling substance for detecting the cell death of the canine tumor-derived cells or the cells which express the canine tumor antigen. Examples of the labeling substance comprise luciferase, a radioisotope and the like.

The “cells in which a canine CD16-canine Fc ε receptor γ chain fusion protein is expressed in human-derived NK cells” refer to cells which function as the effector cells in the “method for measuring antibody-dependent cellular cytotoxicity activity” of the invention.

The “canine CD16-canine Fc ε receptor γ chain fusion protein” means a protein containing the extracellular domain of canine CD16 and canine Fc ε receptor γ chain as an intracellular domain.

The extracellular domain of canine CD16 may be a domain having the amino acid sequence of SEQ ID NO: 1 of the sequence listing or an amino acid sequence in which one or several amino acids have been deleted, substituted or added in the amino acid sequence of SEQ ID NO: 1. The several residues here mean 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3 or 2 residues.

The canine Fc ε receptor γ chain may be one having the amino acid sequence of SEQ ID NO: 2 of the sequence listing or an amino acid sequence in which one or several amino acids have been deleted, substituted or added in the amino acid sequence of SEQ ID NO: 2. The several residues here mean 21, 20, 19, 18, 17, 16, 15, 14, 13, 12, 11, 10, 9, 8, 7, 6, 5, 4, 3 or 2 residues.

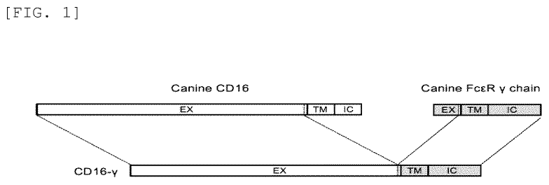

Such a “canine CD16-canine Fc ε receptor γ chain fusion protein” can be expressed by introducing a plasmid containing a gene encoding canine CD16 (FIG. 1, Canine CD16) and a gene encoding canine Fc ε receptor γ chain (FIG. 1, Canine Fc ε R γ chain), as shown in the schematic figure of FIG. 1, into human-derived NK cells.

The human-derived NK cells may be any cells as long as the cells can be cells that function as the effector cells in the “method for measuring antibody-dependent cellular cytotoxicity activity” of the invention. Examples thereof comprise human NK cells such as NK-92 cells, KAI3 cells and KYHG-1 cells, but in particular, NK-92 cells are preferable.

The “antibody” may be any antibody as long as the antibody can be the subject of use in the “method for measuring antibody-dependent cellular cytotoxicity activity” of the invention, but “an antibody for a canine tumor antigen” is preferable. The “antibody” is particularly preferably “an anti-canine CD20 antibody”. The “antibody” may be a commercial product or may be produced independently. An “antibody” which can be used as an antibody drug is particularly preferable.

Examples of the “anti-canine CD20 antibody” comprise 4E1-7-B antibody having the IgG-B constant region of a dog IgG heavy chain, 4E1-7-C antibody having the IgG-C constant region of a dog IgG heavy chain, and afucosylated antibodies thereof, namely 4E1-7-B_f antibody, which are obtained by a similar method to that of Reference Document 1 (JP2021-059499A), and the like. The antibody may also be 4E1-7 antibody, which is obtained by a similar method to that of Reference Document 2 (JP2019-026625A). Furthermore, the “anti-canine CD20 antibody” may be an antibody obtained using silkworm, tobacco or the like or another antibody.

The “cells for measuring antibody-dependent cellular cytotoxicity activity in which a canine CD16-canine Fc ε receptor γ chain fusion protein is expressed in human-derived NK cells” of the invention are the “cells in which a canine CD16-canine Fc ε receptor γ chain fusion protein is expressed in human-derived NK cells” described above which are used for measuring antibody-dependent cellular cytotoxicity activity.

The cells can be obtained by a production method comprising “a step of introducing a gene encoding canine CD16 and a gene encoding canine Fc ε receptor γ chain into human-derived NK cells”. The production method may be a method comprising another step which is useful for producing the cells.

The “cells for measuring antibody-dependent cellular cytotoxicity activity in which a canine CD16-canine Fc ε receptor γ chain fusion protein is expressed in human-derived NK cells” of the invention can be produced, for example, by cloning and amplifying “a gene encoding canine CD16” and “a gene encoding canine Fc ε receptor γ chain”, then inserting the genes into a virus expression plasmid and adding a virus produced by introducing the plasmid into cells to human-derived NK cells.

Here, the “gene encoding canine CD16” may be a gene encoding a protein that has the amino acid sequence of SEQ ID NO: 1 of the sequence listing or an amino acid sequence in which one or several amino acids have been deleted, substituted or added in the amino acid sequence of SEQ ID NO: 1 and that contains the extracellular domain of canine CD16. The several residues here mean 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3 or 2 residues.

Moreover, the “gene encoding canine Fc E receptor γ chain” may be a gene encoding a protein that has the amino acid sequence of SEQ ID NO: 2 of the sequence listing or an amino acid sequence in which one or several amino acids have been deleted, substituted or added in the amino acid sequence of SEQ ID NO: 2 and that contains canine Fc ε receptor γ chain as an intracellular domain. The several residues here mean 21, 20, 19, 18, 17, 16, 15, 14, 13, 12, 11, 10, 9, 8, 7, 6, 5, 4, 3 or 2 residues.

The “cells for measuring antibody-dependent cellular cytotoxicity activity in which canine CD20 and a labeling substance are expressed in lymphoma-derived cells” of the invention are the “cells in which canine CD20 and a labeling substance are expressed in lymphoma-derived cells” described above which are used for measuring antibody-dependent cellular cytotoxicity activity.

The cells can be obtained by a production method comprising “a step of introducing a gene encoding canine CD20 and a gene encoding a labeling substance into lymphoma-derived cells”. The production method may be a method comprising another step which is useful for producing the cells.

The “cells for measuring antibody-dependent cellular cytotoxicity activity in which canine CD20 and a labeling substance are expressed in lymphoma-derived cells” of the invention can be produced, for example, by cloning and amplifying “a gene encoding canine CD20”, inserting the gene into a virus expression plasmid, obtaining a virus produced by introducing the plasmid into cells and adding the virus to lymphoma-derived cells together with a virus produced by introducing a virus expression plasmid containing a gene encoding a labeling substance into cells.

Here, the “gene encoding canine CD20” may be a gene encoding a protein that has the amino acid sequence of SEQ ID NO: 3 of the sequence listing or an amino acid sequence in which one or several amino acids have been deleted, substituted or added in the amino acid sequence of SEQ ID NO: 3 and that contains canine CD20. The several residues here mean 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3 or 2 residues.

Although Examples and the like of the invention are shown below, the invention is not limited thereto only.

### EXAMPLES

### Example 1

Cells in which Canine CD16-Canine Fcs Receptor γ Chain Fusion Protein is Expressed in Human-Derived NK Cells

### 1) Cell Culture

Human NK cell line NK-92 (given by ATCC) was cultured in R10 complete medium (20% FBS and 1000 IU/ml human IL-2 (containing Proleukin (registered trademark) Chiron Therapeutics))) using a humidity incubator (5% CO₂, 37° C.).

### 2) Production of Expression Plasmid

Cloning and production of an expression plasmid were conducted using the primers shown in Table 1. The primers used in this Example were all outsourced.

Canine CD16 was amplified based on CDNA of white blood cells of a healthy dog (beagle) by PCR using primers YTM39 and YTM41, and canine Fcε receptor γ chain was amplified based on CDNA of lymph nodes of a healthy dog by PCR using primers YTM117 and YTM118. The both were cloned to pCR2.1 (registered trademark)-TOPO (registered trademark) vector (Thermo Fisher Scientific), and thus pCR-cCD16 #18 and pCR-cγ #10 were obtained.

After amplifying the amplified canine CD16 again by PCR using primers YTM41 and M13 (−20), the amplified fragment was cloned to the XhoI-SnaBI site of pMx-IP, and thus pMx-IP-cCD16 #1 was obtained. Moreover, pCR-cγ #10 was amplified again by PCR using primers YTM1668 and YTM1669 and then amplified by PCR using primers YTM1668 and YTM838 to add a FLAG tag sequence. The fragment and an amplification product obtained by amplifying pMx-IP-cCD16 #1 by PCR using primers YTM178 and YTM1667 were amplified by overlap PCR using primers YTM178 and YTM838 and then cleaved with NotI. The fragment and SnaBI-NheI fragment encoding IRES-Puro gene cut out of pMx-IP were cloned to the NotI-XbaI site of CSII-EF-MCS-IRES2-Venus vector (RIKEN BioResource Research Center).

As a result, as a lentivirus expression plasmid, CSII-EF-cCD16γ-FL-IP containing a gene encoding a canine CD16-canine Fcε receptor γ chain fusion protein and a puromycin selection gene to which FLAG tag was added at the C terminal was obtained.

TABLE 1

| SEQ | Primer | |
|---|---|---|
| ID NO: | Name | Sequence |
| 4 | YTM39 | CCTTCAGAGCTCGGCATATC |
| 5 | YTM41 | CCGTCGACGGGTTGTGGATCCAGAGAGA |
| 6 | YTM117 | GGGGAACTCTGTGGTCAGG |
| 7 | YTM118 | GAGGGGCAGGAGGTCTTCTA |
| 8 | M13 (−20) | CGACGTTGTAAAACGACGGCCAGT |
| 9 | YTM1668 | CTACTCCTTTCACACCCTCAGCTCTGCTAC |
| 10 | YTM1669 | GTCGATGTCATGATCTTTATAATCCTGGGGGGG |
| | | TTTCTC |
| 11 | YTM838 | TCACTACTTGTCATCGTCATCCTTGTAGTCGAT |
| | | GTCATG |
| 12 | TYM178 | CCACCGCCCTCAAAGTAGACG |
| 13 | YTM1667 | GTAGCAGAGCTGAGGGTGTGAAAGGAGTAG |

### 3) Establishment of Cell Line

Using PEI Max (Polysciences, Inc.), CSII-EF-cCD16γ-FL-IP produced in 2) above was introduced into HEK293T cells into which pCVSVG and p8.9QV were introduced, and the produced lentivirus was added to NK-92 cells. Then, NK-92/cCD16γ cells were obtained through selection in the presence of 0.5 μg/ml puromycin. By subculturing the cells, NK-92/cCD16γ cell line was established as cells in which a canine CD16-canine Fcs receptor γ chain fusion protein was expressed in human-derived NK cells.

### 4) Western Blotting

Cultured NK-92/cCD16γ cell line, which was established in 3) above, was lysed in 1% NP40 buffer. The obtained protein lysate was subjected to SDS-PAGE and western blotting according to normal methods. An anti-Flag M2 antibody (Sigma-Aldrich Japan) and an anti-actin mouse monoclonal antibody (Sigma-Aldrich Japan) were used as primary antibodies, and an HRP-linked antibody (HRP-linked anti-rat Ab (Zymed)) or a goat anti-mouse IgG-HRP (Biorad) was used as a secondary antibody. After the reaction, the membrane was immersed in Western Lightning Plus-ECL reagent (Perkin Elmer), and the results were examined using Luminescent Image Analyzer LAS 3000 mini (FUJIFILM). As a result, a clear band corresponding to the predicted molecular weight of the canine CD16-canine Fcs receptor γ chain fusion protein could be observed.

### Example 2

### Canine Tumor Antigen-Expressing Cells

### 1) A20/cCD20/Luc Cell Line

### (1) Cell Culture

Mouse lymphoma A20 cell line (given by Cell Resource Center for Biomedical Research-Cell Bank) was cultured in R10 complete medium (RPMI1640 medium to which 10% FBS, 100 U/ml penicillin, 100 μg/ml streptomycin and 55 μM 2-mercaptoethanol were added) using a humidity incubator (5% CO₂, 37° C.).

### (2) Production of Expression Plasmids

Cloning and production of expression plasmids were conducted using the primers shown in Table 2. The primers used in this Example were all outsourced.

A luciferase gene was amplified by PCR using primers YTM912 and YTM913, cleaved with AgeI and linked to the HindIII-EcoRV site of pBluescript SK(−) (Promega KK) having the HindIII-AgeI fragment of pGL4.50luc2, and thus pBS-luc2 was obtained. The XhoI fragment and the NotI fragment of pBS-luc2 were inserted into the XhoI-NotI site of pMX-IP (given by Dr. Kurzman (University of Wisconsin)), and thus pMX-luc-IP was obtained as a retrovirus expression plasmid.

A Zeocin resistant gene was amplified using pFUSE-hIgG2-Fc2 (Invivogen) as a template by PCR using primers YTM1931 and YTM1932. The amplification product was cleaved with SalI and NcoI and cloned to the NcoI-SalI site of pMx-IP-cCD20-flag #4, which was produced as follows by a similar method to that of Reference Document 1 (JP2021-059499A), and thus pMx-IZ-cCD20-flag #3 was obtained as a retrovirus expression plasmid.

<pMx-IP-cCD20-Flag #4>

Canine CD20 (sometimes simply referred to as CD20 below) was cloned using uterocervical lymph nodes of a healthy beagle. Specifically, canine CD20 was amplified using a nucleotide sequence of canine CD20 (NCBI registration number: AB210085) as a template by PCR using primers YTM19 and YTM20.

The PCR was conducted using KOD DNA polymerase-Plus (TOYOBO) by denaturation step at 95° C. for two minutes and then 30 cycles of 95° C. for 30 seconds, 56° C. for 30 seconds and 72° C. for 1 to 1.5 minutes. Then, in the presence of Taq DNA polymerase, incubation at 72° C. for 10 minutes was conducted.

The amplification product was linked to a vector using TOPO TA cloning kit (Thermo Fisher Scientific), and thus pCR-cCD20 was obtained. After pCR-cCD20 was amplified by PCR using primers YTM1233 and YTM1234, next PCR was conducted using primers YTM1233 and YTM838. The amplification product was cleaved with BamHI and linked to the BamHI-SnaBI site of pMXs-IP, and thus pMx-IP-cCD20-flag #4 was obtained as a retrovirus expression plasmid.

TABLE 2

| SEQ | Primer | |
|---|---|---|
| ID NO: | Name | Sequence |
| 14 | YTM912 | GCTAAGGTGGTGGACTTGGA |
| 15 | YTM913 | CCGCCCCGACTCTAGAATTA |
| 16 | YTM1931 | AACCATGGCCAAGTTGACCAGTGCCG |
| 17 | YTM1932 | TCGTCGACTCAGTCCTGCTCCTCTGCCACAAAG |
| 18 | YTM19 | GCGCGGCCGCTCTCAGGAGTTCAGAGGGTGAG |
| 19 | YTM20 | CAGAATTCTCAGGAAACAGGGGTGGATA |
| 20 | YTM1233 | ACGGATCCATGACAACACCCAGAAATTCAATG |
| 21 | YTM1234 | GTCGATGTCATGATCTTTATAATCAGGGATGCTGT |
| | | CGT |
| 11 | YTM838 | TCACTACTTGTCATCGTCATCCTTGTAGTCGATGT |
| | | CATG |

### (3) Establishment of Cell Line

Into PLAT-E cell line (Cosmo Bio Co., Ltd.), pMX-luc-IP produced in 1) (2) above was introduced, and the produced retrovirus was added to A20 cells. Furthermore, pMx-IZ-cCD20-flag #3 was introduced into PLAT-E cell line, and the produced retrovirus was added to the A20 cells. The cells were subjected to selection in the presence of 1.0 μg/ml puromycin (FUJIFILM Wako Chemical Corporation) and 300 μg/ml Zeocin (Invivogen), and thus A20/cCD20/luc cells were obtained. The cells were cells expressing canine CD20, and A20/cCD20/luc cell line was established as a cCD20/luc-expressing lymphoma-derived cell line by subculturing the cells.

### 2) EL-4/cCD20/Luc Cell Line

### (1) Cell Culture

Mouse lymphoma EL-4 line (given by Cell Resource Center for Biomedical Research-Cell Bank) was cultured in R10 complete medium using a humidity incubator (5% CO₂, 37° C.).

### (2) Production of Expression Plasmids

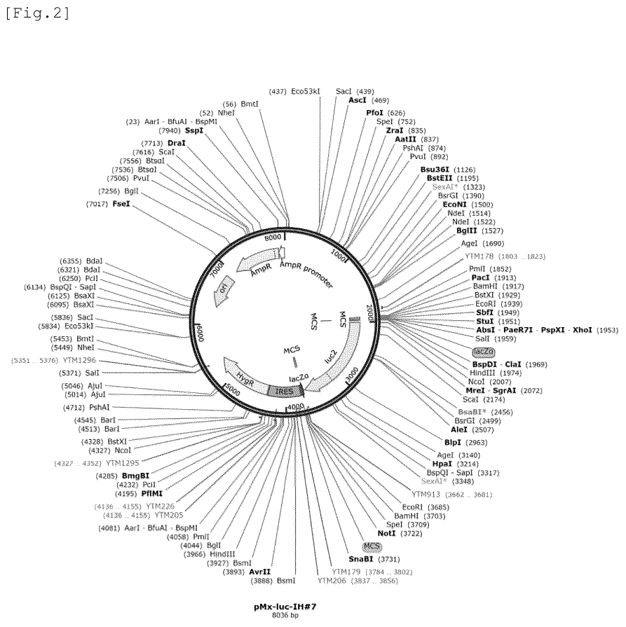

Into the SpeI-EcoRI site of CSII-EF-MCS-IRES2-Venus vector (Riken BioResource Research Center), luc-IRES-Hygromycin which was cut out of pMx-luc-IH vector (FIG. 2) was inserted, and thus CSII-EF-luc-IH #4 was produced. The SpeI fragment and the EcoRI fragment thereof were inserted into the SpeI-EcoRI site of CSII-CMV-MCS-IRES2-Bsd vector (Riken BioResource Research Center), and thus CSII-EF-luc-IB #1 was obtained as a retrovirus expression plasmid. Moreover, pMx-IP-cCD20-flag #4 was obtained by a similar method to that of 1) (2) above.

### (3) Establishment of Cell Line

Into PLAT-E cell line, pMx-cCD20-Flag-IP #4 produced in 2) (2) above was introduced, and the produced retrovirus was added to EL-4 cells. Furthermore, CSII-EF-luc-IB #1 was introduced into PLAT-E cell line, and the produced retrovirus was added to the EL-4 cells. The cells were subjected to selection in the presence of 2.5 μg/ml puromycin and 5 μg/ml Blasticidin S (Kaken Pharmaceutical Co., Ltd.), and thus EL-4/cCD20/luc cells were obtained. The cells were cells expressing canine CD20, and EL-4/cCD20/luc cell line was established as a cCD20/luc-expressing lymphoma-derived cell line by subculturing the cells.

### Example 3

### Measurement Method of ADCC Activity

Antibody-dependent cellular cytotoxicity activity was measured through a step of causing an antibody and cells in which a canine CD16-canine Fc ε receptor γ chain fusion protein was expressed in human-derived NK cells to act on canine tumor-derived cells or cells which expressed a canine tumor antigen.

### 1. Samples

### 1) Cell Lines

As the effector cells, NK-92 cell line or the cells in which a canine CD16-canine Fc ε receptor γ chain fusion protein was expressed in human-derived NK cells and which were established in Example 1 above were used. As the target cells, the cell lines which expressed a canine tumor antigen and which were established in Example 2 above and CLBL-1/luc cell line established as follows by a similar method to that of Reference Document 1 (JP2021-059499A) were used.

### <CLBL-1/luc Cell Line>

The XhoI-NotI fragment of pBS-luc2 plasmid produced in Example 2 above was cut out and inserted into the XhoI-NotI site of pMX-IP, and thus produced pMX-luc-IP #9 was introduced into PLAT-E cell line. The produced retrovirus was added to PG13 cell line (PG13/luc), and the retrovirus produced from the cell line was added to CLBL-1 cells. The cells were subjected to selection in the presence of 0.5 μg/ml puromycin, and thus CLBL-1/luc cells were obtained. The cells were subcultured in R10 complete medium using a humidity incubator (5% CO₂, 37° C.), and thus CLBL-1/luc cell line was established.

### 2) Antibodies

(1) Rat IgG2a Antibody (Ebioscience, Inc.)

(2) Dog IgG Antibody (Jackson ImmunoResearch Laboratories, Inc.)

(3) 4E1-7-B Antibody

By a similar method to that of Reference Document 1 (JP2021-059499A), 4E1-7-B antibody, which is a monoclonal antibody for canine CD20 and has the IgG-B constant region of a dog IgG heavy chain, was obtained. This 4E1-7-B antibody was a monoclonal antibody for canine CD20 having a heavy-chain variable region, a light-chain variable region, a light-chain constant region and a heavy-chain constant region having the amino acid sequences of A to D below.

A. A heavy-chain variable region having the amino acid sequence of SEQ ID NO: 22 or an amino acid sequence in which one or several amino acids have been deleted, substituted or added in the amino acid sequence of SEQ ID NO: 22.

The several residues here mean 28, 27, 26, 25, 24, 23, 22, 21, 20, 19, 18, 17, 16, 15, 14, 13, 12, 11, 10, 9, 8, 7, 6, 5, 4, 3 or 2 residues.

B. A light-chain variable region having the amino acid sequence of SEQ ID NO: 23 or an amino acid sequence in which one or several amino acids have been deleted, substituted or added in the amino acid sequence of SEQ ID NO: 23.

The several residues here mean 27, 26, 25, 24, 23, 22, 21, 20, 19, 18, 17, 16, 15, 14, 13, 12, 11, 10, 9, 8, 7, 6, 5, 4, 3 or 2 residues.

C. A light-chain constant region having the amino acid sequence of SEQ ID NO: 24 or an amino acid sequence in which one or several amino acids have been deleted, substituted or added in the amino acid sequence of SEQ ID NO: 24.

The several residues here mean 22, 21, 20, 19, 18, 17, 16, 15, 14, 13, 12, 11, 10, 9, 8, 7, 6, 5, 4, 3 or 2 residues.

D. A heavy-chain constant region having the amino acid sequence of SEQ ID NO: 25 or an amino acid sequence in which one or several amino acids have been deleted, substituted or added in the amino acid sequence of SEQ ID NO: 25.

The several residues here mean 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3 or 2 residues.

2. Measurement of ADCC Activity Target cells (A20/cCD20/luc cell line, EL-4/cCD20/luc cell line or CLBL-1/luc cell line) at 5×10³ cells/well were seeded to a 96-well microtiter plate containing an antibody (the rat IgG2a antibody, the dog IgG antibody or 4E1-7-B antibody). The plate was incubated on ice for 20 minutes, and then NK-92 cells or NK-92/cCD16γ cell line in a total volume of 100 μL was added as the effector cells at an effector cell/target cell ratio of 10:1 (E/T of 10:1).

The cultures were further incubated for four hours, and then the cells were lysed using ONE-Glo luciferase assay system (Promega KK). The luciferase activities were detected by ARVO X4 system (PerkinElmer).

The cell-mediated cytotoxicity activity (lysis rate) was calculated by the following equation from the relative luminometer unit (RLU) data. The lysis rate was calculated from the average of three wells.

Specific Lysis (%)=100×(Spontaneous Death RLU−Test RLC)/(Spontaneous Death RLU−Maximal Killing RLU) [Math. 1]

### 3. Results

### 1) When CLBL-1/luc Cell Line was Used as Target Cells

When NK-92 cell line was used as the effector cells, high cell-mediated cytotoxicity activity (lysis rate) could be observed both in the case without using the antibodies and in the case using the rat IgG 2a antibody or 4E1-7-B antibody.

When NK-92/cCD16γ cell line was used as the effector cells, there was a tendency towards a slight increase in the cell-mediated cytotoxicity activity (lysis rate) when 4E1-7-B antibody was used compared to the case without using the antibodies or the case using the rat IgG 2a antibody, but there was no large difference.

Accordingly, it was shown that CLBL-1/luc cell line is not suitable for measurement of ADCC activity using NK-92 cell line or a cell line derived from the NK-92 cell line because the sensitivity as the target cells is too high.

2) When Canine Tumor Antigen-Expressing Cell Lines were Used as Target Cells

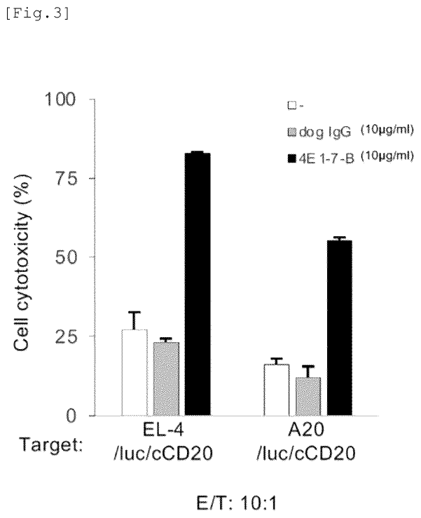

When NK-92/cCD16γ cell line was used as the effector cells, the cell-mediated cytotoxicity activity (lysis rate) was around 25% at the highest in the case without using the antibodies (FIG. 3, −(none)) or the case using the dog IgG antibody as the antibody (FIG. 3, dog IgG), in both target cells, while cell-mediated cytotoxicity activity (lysis rate) of 50% or more could be observed in the case using 4E1-7-B antibody (FIG. 3, 4E1-7-B), which is a monoclonal antibody for canine CD20.

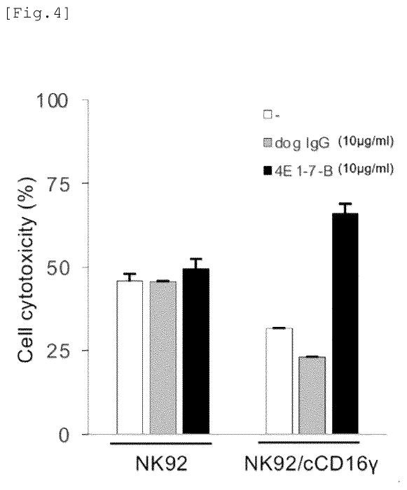

Moreover, when A20/cCD20/luc cell line was used as the target cells and NK-92 cell line was used as the effector cell line, there was no large difference in the cell-mediated cytotoxicity activity (lysis rate) (FIG. 4, NK92) among the case without using the antibodies (FIG. 4, −(none)) and the cases using the dog IgG antibody (FIG. 4, dog IgG) or 4E1-7-B antibody (FIG. 4, 4E1-7-B). When NK-92/cCD16γ cell line was used as the effector cells, however, particularly high cell-mediated cytotoxicity activity (lysis rate) could be observed in the case using 4E1-7-B antibody (FIG. 4, NK92/cCD16γ).

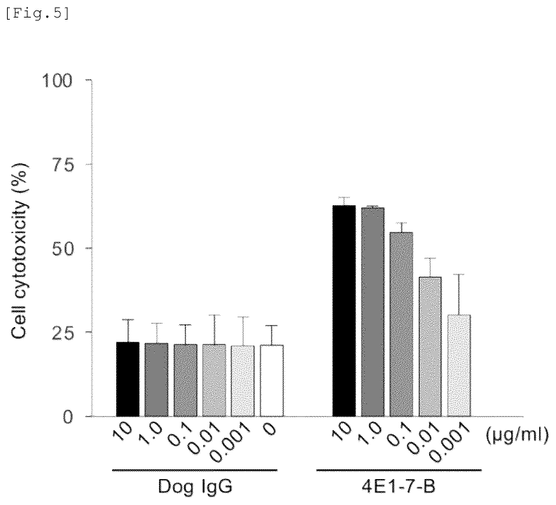

Furthermore, the 4E1-7-B antibody concentration-dependent effects were examined using A20/cCD20/luc cell line as the target cells and using NK-92 cell line as the effector cell line. As a result, cytotoxic effects were observed with the dog IgG antibody (FIG. 5, dog IgG) regardless of the concentration, while cell death could be induced in a concentration dependent manner with 4E1-7-B antibody (FIG. 5, 4E1-7-B).

Accordingly, the results show that, when canine tumor-derived cells or cells which express a canine tumor antigen are used as the target cells and cells in which a canine CD16-canine Fc ε receptor γ chain fusion protein is expressed in human-derived NK cells are used as the effector cells, a low-variation and highly reproducible method for measuring antibody-dependent cellular cytotoxicity activity can be provided.

Moreover, it was also found that 4E1-7-B antibody, which is a monoclonal antibody for canine CD20, is an antibody that has high antibody-dependent cellular cytotoxicity activity and that is useful as an antibody drug.

### Example 4

ADCC activity was measured by a similar measurement method to that of Example 3 except for using the antibodies below as the anti-canine CD20 antibodies. A20/cCD20/luc cell line was used as the target cells, and NK-92/cCD16γ cell line was used as the effector cells.

The anti-canine CD20 antibodies were all monoclonal antibodies for canine CD20 having a heavy-chain variable region, a light-chain variable region, a light-chain constant region and a heavy-chain constant region having the amino acid sequences of A to D shown in Example 3, like 4E1-7-B antibody.

### <Antibodies>

### 1. 4E1-7-B_f Antibody

An afucosylated anti-canine CD20 antibody was obtained by a similar method to that of Reference Document 1 (JP2021-059499A).

### 2. 4E1-7-B_s_m Antibody

An afucosylated anti-canine CD20 antibody was obtained using a silkworm having no fucose transferase.

### 3. Anti-Canine CD20 Antibody Produced Using Tobacco

A gene was introduced with Agrobacterium using tobacco which was genetically modified to lack fucose transferase, and an afucosylated anti-canine CD20 antibody was obtained.

### Measurement of ADCC Activity

Using 4E1-7-B_f antibody, eight individual tests were conducted in which various parameters (the cell concentration, the dosage range, the incubation period, the assay reagent and the like) were adjusted. As a result, the coefficient of variation (CV value) of the cell-mediated cytotoxicity activity (lysis rate) calculated from the relative luminometer unit (RLU) data among the repeated tests was 25% or less. Accordingly, it was found from the results that the method for measuring ADCC activity of the invention is a low-variation and highly reproducible method having high performance as a bioassay.

Furthermore, in experiment with an effector cell/target cell ratio of 10:1 (E/T=10:1), the CV value among the repeated tests was 10% or less, and it was found that the method was most suitable as a method for measuring ADCC activity. In the experiment, the dog IgG used as the negative control did not show reaction, and thus clear specificity was also observed.

Moreover, when ADCC activity was measured using 4E1-7-B_f antibody of another lot (called 4E1-7-B_f No. 2), 4E1-7-B_s_m antibody and the anti-canine CD20 antibody produced using tobacco, ADCC activity which was comparable to that of 4E1-7-B_f antibody used for comparison (called 4E1-7-B_f No. 1) could be observed.

Accordingly, it was found from the results that the method for measuring ADCC activity of the invention is a low-variation and highly reproducible method having high performance as a bioassay in which various anti-canine CD20 antibodies can be used.

### INDUSTRIAL APPLICABILITY

According to the invention, a low-variation and highly reproducible method for measuring antibody-dependent cellular cytotoxicity activity also of an antibody for a dog as the subject of administration can be provided. Moreover, when the measurement method of the invention is used, an antibody drug which is useful for tumor therapy for a dog as the subject of administration is also provided easily.

### SEQUENCE LISTING

1. A method for measuring antibody-dependent cellular cytotoxicity activity comprising:
a step of causing an antibody and cells in which a canine CD16-canine Fcs receptor γ chain fusion protein is expressed in human-derived NK cells to act on canine tumor-derived cells or cells which express a canine tumor antigen; and
a step of detecting cell death of the canine tumor-derived cells or the cells which express the canine tumor antigen.

2. The measurement method according to claim 1, wherein the human-derived NK cells are NK-92 cells.

3. The measurement method according to claim 1, wherein the cells which express the canine tumor antigen are lymphoma-derived cells containing a labeling substance.

4. The measurement method according to claim 1, wherein the canine tumor antigen is CD20.

5. The measurement method according to claim 3, wherein the labeling substance is luciferase.

6. The measurement method according to claim 3, wherein the lymphoma-derived cells are A20 cells or EL-4 cells.

7. The measurement method according to claim 1, wherein the antibody is an antibody for the canine tumor antigen and is an antibody having antibody-dependent cellular cytotoxicity activity.

8. The measurement method according to claim 7, wherein the antibody is an anti-canine CD20 antibody.

9. Cells for measuring antibody-dependent cellular cytotoxicity activity in which a canine CD16-canine Fcε receptor γ chain fusion protein is expressed in human-derived NK cells.

10. A method for producing the cells according to claim 9, comprising a step of introducing a gene encoding canine CD16 and a gene encoding canine Fcε receptor γ chain into the human-derived NK cells.

11. Cells for measuring antibody-dependent cellular cytotoxicity activity in which canine CD20 and a labeling substance are expressed in lymphoma-derived cells.

12. A method for producing the cells according to claim 11, comprising a step of introducing a gene encoding canine CD20 and a gene encoding the labeling substance into the lymphoma-derived cells.

13. The measurement method according to claim 2, wherein the cells which express the canine tumor antigen are lymphoma-derived cells containing a labeling substance.


SEQUENCE LISTING (25 sequences)

SEQ ID NO: 1 - protein, 163 aa, Canine
MDSVTLKCQDHLLRDNYTWLHNGRPISNQISTYIIKNASIKNSGEYRCQTDQSKLSDPVQLEVHTGWLLLQVPRLVFQEGELIQLKCHSWKNTPVRNVQYFQNGRGKKFFYNNSEYHIPAATSEHNGSYFCRGIIGKKNESSEAVNIIIQGSSLPSTSLLLSH

SEQ ID NO: 2 - protein, 104 aa, Canine
PQLCYILDAILFLYGIVLTLLYCRLKIQVRKAAVTSYEKPDGIYTGLSTRNQETYETLKHEKPPQMWQLVSSTALLLLVSAGTQAADVPKAVVVLEPKWNRVLT

SEQ ID NO: 3 - protein, 297 aa, Canine
MTTPRNSMSGTLPVDPMKSPTAMYPVQKIIPKRMPSVVGPTQNFFMRESKTLGAVQIMNGLFHIALGSLLMIHTDVYAPICITMWYPLWGGIMFIISGSLLAAADKNPRKSLVKGKMIMNSLSLFAAISGIIFLIMDIFNITISHFFKMENLNLIKAPMPYVDIHNCDPANPSEKNSLSIQYCGSIRSVFLGVFAVMVIFTFFQKLVTAGIVENEWKKLCSKPKSDVVVLLAAEEKKEQPIETTEEMVELTEIASQPKKEEDIEIIPVQEEEEELEINFAEPPQEQESSPIENDSIP

SEQ ID NO: 4 - DNA, 20 bp, synthetic construct
  source 1..20: mol_type = other DNA
ccttcagagctcggcatatc

SEQ ID NO: 5 - DNA, 28 bp, synthetic construct
  source 1..28: mol_type = other DNA
ccgtcgacgggttgtggatccagagaga

SEQ ID NO: 6 - DNA, 19 bp, synthetic construct
  source 1..19: mol_type = other DNA
ggggaactctgtggtcagg

SEQ ID NO: 7 - DNA, 20 bp, synthetic construct
  source 1..20: mol_type = other DNA
gaggggcaggaggtcttcta

SEQ ID NO: 8 - DNA, 24 bp, synthetic construct
  source 1..24: mol_type = other DNA
cgacgttgtaaaacgacggccagt

SEQ ID NO: 9 - DNA, 30 bp, synthetic construct
  source 1..30: mol_type = other DNA
ctactcctttcacaccctcagctctgctac

SEQ ID NO: 10 - DNA, 39 bp, synthetic construct
  source 1..39: mol_type = other DNA
gtcgatgtcatgatctttataatcctgggggggtttctc

SEQ ID NO: 11 - DNA, 39 bp, synthetic construct
  source 1..39: mol_type = other DNA
tcactacttgtcatcgtcatccttgtagtcgatgtcatg

SEQ ID NO: 12 - DNA, 21 bp, synthetic construct
  source 1..21: mol_type = other DNA
ccaccgccctcaaagtagacg

SEQ ID NO: 13 - DNA, 30 bp, synthetic construct
  source 1..30: mol_type = other DNA
gtagcagagctgagggtgtgaaaggagtag

SEQ ID NO: 14 - DNA, 20 bp, synthetic construct
  source 1..20: mol_type = other DNA
gctaaggtggtggacttgga

SEQ ID NO: 15 - DNA, 20 bp, synthetic construct
  source 1..20: mol_type = other DNA
ccgccccgactctagaatta

SEQ ID NO: 16 - DNA, 26 bp, synthetic construct
  source 1..26: mol_type = other DNA
aaccatggccaagttgaccagtgccg

SEQ ID NO: 17 - DNA, 33 bp, synthetic construct
  source 1..33: mol_type = other DNA
tcgtcgactcagtcctgctcctctgccacaaag

SEQ ID NO: 18 - DNA, 32 bp, synthetic construct
  source 1..32: mol_type = other DNA
gcgcggccgctctcaggagttcagagggtgag

SEQ ID NO: 19 - DNA, 28 bp, synthetic construct
  source 1..28: mol_type = other DNA
cagaattctcaggaaacaggggtggata

SEQ ID NO: 20 - DNA, 32 bp, synthetic construct
  source 1..32: mol_type = other DNA
acggatccatgacaacacccagaaattcaatg

SEQ ID NO: 21 - DNA, 38 bp, synthetic construct
  source 1..38: mol_type = other DNA
gtcgatgtcatgatctttataatcagggatgctgtcgt

SEQ ID NO: 22 - protein, 137 aa, Canine
MKLRLSLIFICALLKDVQCEVQLVESGGGLVQPGASLKLSCVASGFTFNDYWMSWVRQTPGKTMEWIGDIKYDGSYTNYAPSLKNRFTISRDNAKSTLYLQMSNVRSEDTATYYCTREAYYYSGDYWGQGVMVTVSS

SEQ ID NO: 23 - protein, 133 aa, Canine
MKTDTLLLWALLLWVPGCTGDIVLTQSPALAVSLGQRATISCKTNQNVDYYGNSYMHWYQQKPGQQPKLLIYLASNLASGIPARFSGRGSGTDFTLTIDPVEADDTATYYCQQSRNLPYTFGAGTKLELKRND

SEQ ID NO: 24 - protein, 110 aa, Canine
RNDAQPAVYLFQPSPDQLHTGSASVVCLLNSFYPKDINVKWKVDGVIQDTGIQESVTEQDKDSTYSLSSTLTMSSTEYLSHELYSCEITHKSLPSTLIKSFQRSECQRVD

SEQ ID NO: 25 - protein, 334 aa, Canine
STTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK